(19) United States
(12) Patent Application Publication
(10) Pub. No.: US 2024/0226220 A9
(43) Pub. Date: Jul. 11, 2024

(54) ORAL BOTANICAL COMPOSITION

(71) Applicants: ARIZONA BOARD OF REGENTS ON BEHALF OF ARIZONA STATE UNIVERSITY, Scottsdale, AZ (US); SOUTHWEST COLLEGE OF NATUROPATHIC MEDICINE, Tempe, AZ (US)

(72) Inventors: Jeffrey Olaf Langland, Chandler, AZ (US); Bertram Jacobs, Tempe, AZ (US)

(21) Appl. No.: 18/277,043

(22) PCT Filed: Feb. 11, 2022
(86) PCT No.: PCT/US2022/016168

§ 371 Date: Aug. 11, 2023

Related U.S. Application Data
(60) Provisional application No. 63/148,869, filed on Feb. 12, 2021.

(51) Int. Cl. A61K 36/539; A61K 36/287; A61K 36/537; A61K 36/78; A61K 36/82; A61K 36/888; A61P 31/14

(52) U.S. Cl. CPC A61K 36/539; A61K 36/287; A61K 36/537; A61K 36/78; A61K 36/82; A61K 36/888; A61P 31/14; A61K 2236/333

(57) ABSTRACT

This invention is directed to oral botanical compositions comprising plant extracts, methods of preparing the same, and methods of using the same for treating an infection.

This application claims priority from U.S. Provisional Application Ser. No. 63/148,869 filed on Feb. 12, 2021, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to oral botanical compositions comprising plant extracts, methods of preparing the same, and methods of using the same for treating an infection.

### BACKGROUND OF THE INVENTION

Coronavirus outbreaks represent a significant threat to the human population. Previous outbreaks with SARS-CoV-1 and MERS and more recently, SARS-CoV-2 illustrate the concerns over this virus family. Therapeutics to these viral infections remain limited.

### SUMMARY OF THE INVENTION

Aspects of the invention are drawn to an oral botanical composition comprising plant extracts from an Arum genera plant, Houttuynia cordata, and Scutellaria baicalensis.

In embodiments, the Arum genera plant comprises Arum palaestinum, Arum concinnatum, Arum cyrenalcom, Arum dioscoridis, Arum nigrum, or a combination thereof. For example, the Arum genera plant can be selected from the group consisting of Arum concinnatum, Arum cyrenalcom, Arum dioscoridis, and Arum nigrum.

Embodiments can further comprise a plant extract from Chrysanthemum indicum, Camellia sinensis, Salvia miltiorrhiza, or a combination thereof.

In embodiments, the plant extract is an alcoholic extract. For example, the alcoholic extract is a 20% ethanol extract or a 70% ethanol extract.

In embodiments, the plant extract comprises the root or aerial parts of the Arum genera plant. For example, the plant extract comprises the root or aerial parts of Arum palaestinum. For example, the plant extract comprises the root parts of Arum concinnatum, Arum cyrenalcom, Arum dioscoridis, or Arum nigrum.

In embodiments, the plant extract comprises the leaf parts of Camellia sinensis.

In embodiments, the plant extract comprises the flower parts of Chrysanthemum indicum.

In embodiments, the plant extract comprises the root parts of Salvia miltiorrhiza.

In embodiments, the Arum genera plant is present in an amount ranging from about 1 to about 20 wt % of the total composition.

In embodiments, the Houttuynia cordata is present in an amount ranging from about 5 to about 20 wt % of the total composition.

In embodiments, the Scutellaria baicalensis is present in an amount ranging from about 30 to about 70 wt % of the total composition.

In embodiments, the Camellia sinensis is present in an amount ranging from about 15 to about 25 wt % of the total composition.

In embodiments, the Chrysanthemum indicum is present in an amount ranging from about 50 to about 60 wt % of the total composition.

In embodiments, the Salvia miltiorrhiza is present in an amount ranging from about 30 to about 40 wt % of the total composition.

In embodiments, the plant extract from the Arum genera plant, Houttuynia cordata, and Scutellaria baicalensis are in a ratio of about 1:3:3.

Aspects of the invention are also drawn to an oral botanical composition comprising plant extracts from Artemisia annua, Salvia miltiorrhiza, Houttuynia cordata, Isatis indigotica, Bupleurum chinense, Cinnamomum cassia, Citrus reticulata, Glycyrrhiza uralensis, Coptis spp., Zingiber officinale, or any combination thereof. In embodiments, Houttuynia cordata is replaced with Scutellaria baicalensis.

In embodiments, the Artemisia annua is present in an amount ranging from about 15 to about 40 wt % of the total composition.

In embodiments, the Salvia miltiorrhiza is present in an amount ranging from about 1 to about 30 wt % of the total composition.

In embodiments, the Isatis indigotica is present in an amount ranging from about 5 to about 30% wt of the total composition.

In embodiments, the Bupleurum chinense is present in an amount ranging from about 1 to about 30 wt % of the total composition.

In embodiments, the Cinnamomum cassia is present in an amount ranging from about 0.5 to about 10% wt of the total composition.

In embodiments, the Glycyrrhiza uralensis is present in an amount ranging from about 1 to about 20% wt of the total composition.

In embodiments, the Coptis spp. is present in an amount ranging from about 0.25 to about 20% wt of the total composition.

In embodiments, the Zingiber officinale is present in an amount ranging from about 0.25 to about 10% wt of the total composition.

In embodiments, the botanical composition can comprise plant extracts from Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale. For example, the botanical composition can comprise Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale in a ratio of 12:8:6:4:4:4:4:1:1. For example, the botanical composition can comprise Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale in a ratio of 19:4:11:11:16:4:11:11:11:2. For example, the botanical composition can comprise Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale in a ratio of 31:6:19:19:5:5:5:5:2.5:2.5. In embodiments, Houttuynia cordata is replaced with Scutellaria baicalensis. In embodiments, the composition is in a form selected from the group consisting of a liquid, a gummy, a tablet, a tincture a pellet, a lozenge, a granule, a suspension, and a capsule.

In embodiments, the composition is synergistic.

Aspects of the invention are further drawn to an aqueous botanical extract composition, such as a composition comprising plant extracts wherein the composition comprises about 1-20% extract of Arum genera plant, about 5-20% Houttuynia cordata, and/or about 30-70% Scutellaria baicalensis.

Embodiments can further comprise about 50-60% extract of Chrysanthemum indicum, about 15-25% extract of Camellia sinensis, and/or about 30-40% Salvia miltiorrhiza.

In embodiments, the Arum genera plant comprises Arum palaestinum, Arum concinnatum, Arum cyrenalcom, Arum dioscoridis, Arum nigrum, or a combination thereof. For example, the Arum genera plant is selected from the group consisting of Arum concinnatum, Arum cyrenalcom, Arum dioscoridis, and Arum nigrum.

In embodiments, the plant extract is an alcoholic extract. For example, the alcoholic extract is a 20% ethanol extract or a 70% ethanol extract.

In embodiments, the plant extract comprises the root or aerial parts of the Arum genera plant. For example, the plant extract comprises the root or aerial parts of Arum palaestinum. For example, the plant extract comprises the root parts of Arum concinnatum, Arum cyrenalcom, Arum dioscoridis, or Arum nigrum.

In embodiments, the plant extract comprises the leaf parts of Camellia sinensis.

In embodiments, the plant extract comprises the flower parts of Chrysanthemum indicum.

In embodiments, the plant extract comprises the root parts of Salvia miltiorrhiza.

In embodiments, the aqueous botanical extract composition is synergistic.

Still further, aspects of the invention are drawn to a therapeutic botanical preparation. For example, the therapeutic botanical preparation can comprise plant extracts from an Arum genera plant, Houttuynia cordata, and Scutellaria baicalensis, and at least one pharmaceutically acceptable excipient.

In embodiments, the Arum genera plant comprises Arum palaestinum, Arum concinnatum, Arum cyrenalcom, Arum dioscoridis, Arum nigrum, or a combination thereof. For example, the Arum genera plant is selected from the group consisting of Arum concinnatum, Arum cyrenalcom, Arum dioscoridis, and Arum nigrum.

Embodiments can further comprise a plant extract from Chrysanthemum indicum, Camellia sinensis, Salvia miltiorrhiza, or a combination thereof.

In embodiments, the plant extract is an alcoholic extract. For example, the alcoholic extract is a 20% ethanol extract or a 70% ethanol extract.

In embodiments, the plant extract comprises the root or aerial parts of the Arum genera plant. For example, the plant extract comprises the root or aerial parts of Arum palaestinum. For example, the plant extract comprises the root parts of Arum concinnatum, Arum cyrenalcom, Arum dioscoridis, or Arum nigrum.

In embodiments, the plant extract comprises the leaf parts of Camellia sinensis.

In embodiments, the plant extract comprises the flower parts of Chrysanthemum indicum.

In embodiments, the plant extract comprises the root parts of Salvia miltiorrhiza.

In embodiments, the Arum genera plant is present in an amount ranging from about 1 to about 20 wt % of the total composition.

In embodiments, the Houttuynia cordata is present in an amount ranging from about 5 to about 20 wt % of the total composition.

In embodiments, the Scutellaria baicalensis is present in an amount ranging from about 30 to about 70 wt % of the total composition.

In embodiments, the Camellia sinensis is present in an amount ranging from about 15 to about 25 wt % of the total composition.

In embodiments, the Chrysanthemum indicum is present in an amount ranging from about 50 to about 60 wt % of the total composition.

In embodiments, the Salvia miltiorrhiza is present in an amount ranging from about 30 to about 40 wt % of the total composition.

In embodiments, the composition is in a form selected from the group consisting of a liquid, a gummy, a tablet, a tincture a pellet, a lozenge, a granule, a suspension, and a capsule.

In embodiments, the composition is synergistic.

Also, aspects of the invention are drawn to methods for preparing a liquid extract from a plant material. For example, the plant material can be selected from an Arum genera plant, Houttuynia cordata, Scutellaria baicalensis, Chrysanthemum indicum, Camellia sinensis, and Salvia miltiorrhiza.

In embodiments, the method comprises obtaining fresh plant material; washing and air drying the plant material; grinding the plant material to a powder; combining the powder with a liquid comprising at least one of water and ethanol to concentrate the solution to obtain an alcohol extract of the plant materials; extracting plant material by liquid at about 20° C. to about 37° C. to form a liquid extract; and separating the liquid extract from the plant material.

In embodiments, the Arum genera plant comprises Arum palaestinum, Arum concinnatum, Arum cyrenalcom, Arum dioscoridis, Arum nigrum, or a combination thereof.

In embodiments, the plant extract is an alcoholic extract. For example, the alcoholic extract is a 20% ethanol extract or a 70% ethanol extract.

In embodiments, the plant extract comprises the root or aerial parts of the Arum genera plant. For example, the plant extract comprises the root or aerial parts of Arum palaestinum. For example, the plant extract comprises the root parts of Arum concinnatum, Arum cyrenalcom, Arum dioscoridis, or Arum nigrum.

In embodiments, the plant extract comprises the leaf parts of Camellia sinensis.

In embodiments, the plant extract comprises the flower parts of Chrysanthemum indicum.

In embodiments, the plant extract comprises the root parts of Salvia miltiorrhiza.

In embodiments, the Arum genera plant is present in an amount ranging from about 1 to about 20 wt % of the total composition.

In embodiments, Houttuynia cordata is present in an amount ranging from about 5 to about 20 wt % of the total composition.

In embodiments, Scutellaria baicalensis is present in an amount ranging from about 30 to about 70 wt % of the total composition.

In embodiments, Camellia sinensis is present in an amount ranging from about 15 to about 25 wt % of the total composition.

In embodiments, Chrysanthemum indicum is present in an amount ranging from about 50 to about 60 wt % of the total composition.

In embodiments, Salvia miltiorrhiza is present in an amount ranging from about 30 to about 40 wt % of the total composition.

In embodiments, the extract is synergistic.

Embodiments also comprise a therapeutic composition comprising at least one liquid extract as described herein.

Embodiments also comprise an aqueous botanical extract composition, wherein each of the extracts are prepared by the methods described herein. For example, the aqueous composition can be an oral composition.

Aspects of the invention are also drawn to methods of decreasing replication of a coronavirus. For example, embodiments comprise exposing the coronavirus to a botanical composition as described herein or a therapeutic botanical preparation as described herein.

Aspects of the invention are further drawn to methods of treating a coronavirus infection in a subject in need thereof. In embodiments, the method comprises administering to the subject a botanical composition as described herein or a therapeutic botanical preparation as described herein.

Further, aspects of the invention are drawn to methods for decreasing coronavirus infection or reducing a symptom thereof in a subject in need thereof. In embodiments, the method comprises administering to a subject in need thereof an effective amount of the botanical composition as described herein or a therapeutic botanical preparation as described herein.

Still further, aspects of the invention are drawn to methods for preventing, treating or improving a coronavirus associated disease or condition. In embodiments, the method comprises administering to a subject in need thereof an effective amount of the botanical composition as described herein or a therapeutic botanical preparation as described herein.

In embodiments, the coronavirus associate disease or condition comprises lung pathology, acute respiratory distress syndrome, pneumonia, organ failure, heart failure, blood clots, acute kidney injury, and/or additional viral or bacterial infections.

In embodiments, the coronavirus comprises human coronavirus NL63 (HCoV-NL63), severe acute respiratory syndrome coronavirus (SARS-CoV), Middle East respiratory syndrome coronavirus (MERS-CoV), or severe acute respiratory syndrome coronavirus-2 (SARS-CoV-2).

In embodiments, the botanical composition is administered prior to or after exposure to the coronavirus.

In embodiments, the botanical composition is administered at a dose sufficient to decrease the viral activity of the coronavirus.

In embodiments, administering comprises oral administration.

Other objects and advantages of this invention will become readily apparent from the ensuing description.

### BRIEF DESCRIPTION OF THE FIGURES

FIG. 1 shows the botanical species tested for anti-coronavirus activity.

FIG. 2 shows example of SARS-CoV-2 CPE inhibition assay.

FIG. 3A shows anti-SARS-CoV-2 activity of effective botanical extracts.

FIG. 3B shows anti-SARS-CoV-2 activity of different Arum spp. extracts.

FIG. 4 shows anti-MHV activity of SARS-CoV-2 botanical extracts.

FIG. 5 shows synergistic anti-SARS-CoV-2 activity of botanical extract combinations.

FIG. 6 shows non-limiting, exemplary formulations of botanical extracts.

### DETAILED DESCRIPTION OF THE INVENTION

Therapeutics for the treatment of coronavirus infections are currently limited and often are directed toward supportive care for immune support or other physiological responses. Even fewer drugs are available which directly target the virus replication process. Our work has identified botanical extracts with anti-viral activity, for example anti-viral activities against coronaviruses, such as SARS-CoV-2 and MHV coronaviruses. Therapeutic combinations of these botanical extracts demonstrated positive synergistic activity related to inhibition of the virus. These synergistic botanical extract formulations can have therapeutic value in preventing and/or treating SARS-CoV-2 and other coronavirus infections. These synergistic botanical formulations can be developed into dietary supplements for the treatment of viral infections, such as corona virus infections. In addition, these extracts can offer a resource for the isolation of new anti-coronavirus compounds.

Detailed descriptions of one or more embodiments are provided herein. However, the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Botanical Compositions

Aspects of the invention are drawn to botanical compositions, such as those comprising plant or botanical extracts from an Arum genera plant, Houttuynia cordata, and Scutellaria baicalensis.

“Botanical” can refer to a material that is or can be obtained from a tree-, plant-, weed- or herb-derived. As used herein, “botanically derived” can refer to a material capable of having been derived from a botanical, as by isolation or extraction; however, “botanically derived” is not limited in this application to materials which actually are isolated or extracted from a botanical, but also includes materials obtained commercially or synthetically.

The term “botanical composition” can refer to a composition that contains vegetable matter, which can derive from any part of a plant or plant material, or combinations thereof. Depending in part on its intended use, a botanical composition can be a food, drug, medical food, dietary supplement, medicinal supplement, nutritional supplement, nutritional or nutrient. The botanical composition can include a number of additional agents.

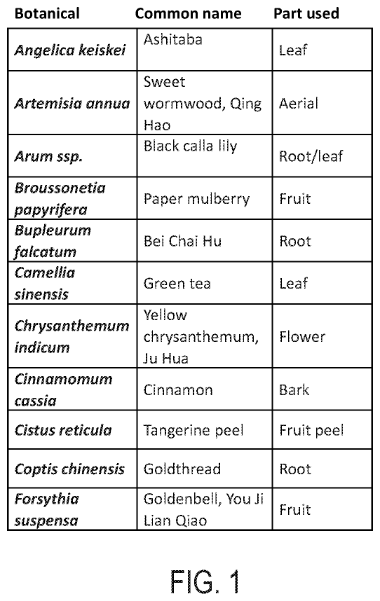

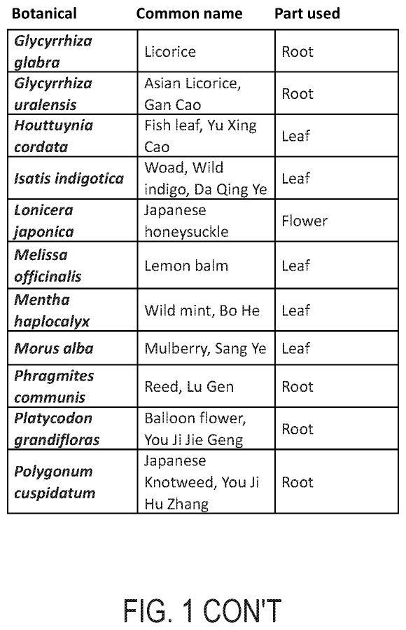

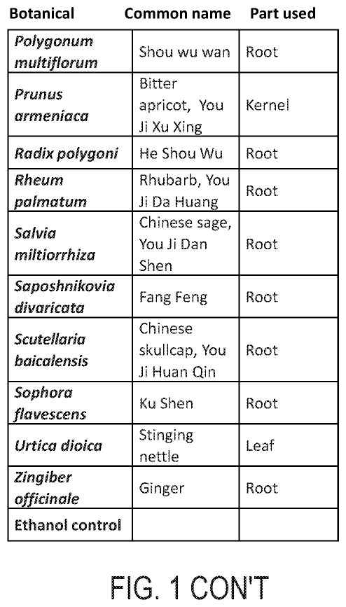

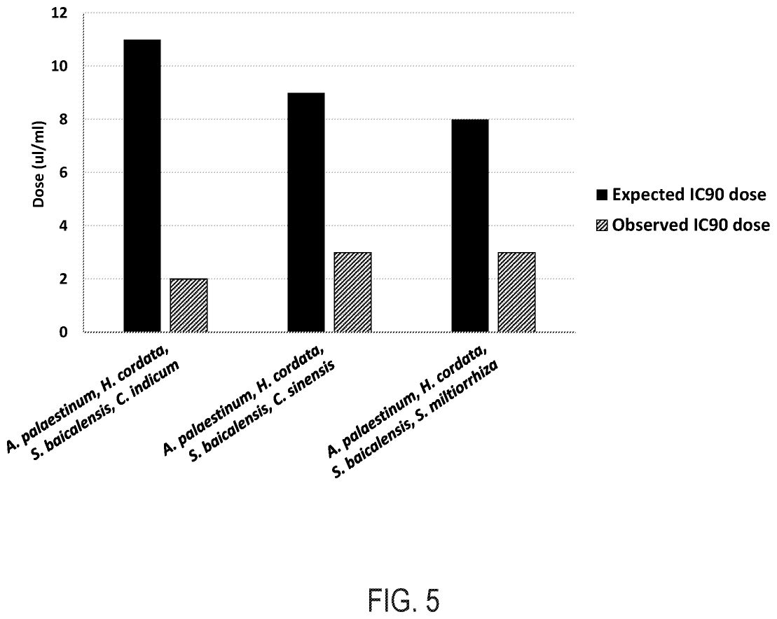

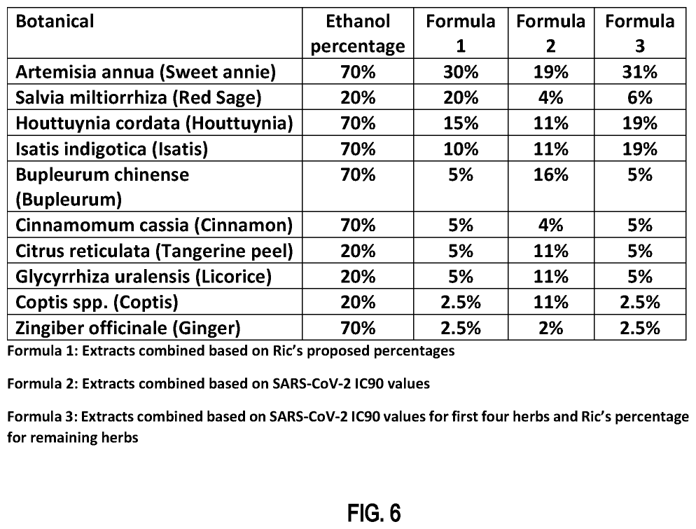

In embodiments, the botanical composition can comprise a combination of plant materials (i.e., a combination of plant extracts). For example, such “combination composition” can refer to a botanical composition which comprises a mixture of at least two different plant materials. In embodiments, the at least two different plant materials can be from the same plant (such as a mixture comprising a first material from an aerial part of the plant and a second material from the root of the plant). In other embodiments, the at least two different plant materials can be from different plants (such as a mixture comprising a first material from a first plant, and a second material from a second plant). For example, the plant materials can be plant extracts, as described herein. In embodiments, the combination composition can comprise a mixture of plant materials from two or more of the plant species, such as those listed in FIG. 1 or FIG. 6. For example, the combination composition can comprise a mixture of plant materials comprising Arum genera (e.g., Arum palaestinum), Houttuynia cordata, Scutellaria baicalensis, Chrysanthemum indicum, Camellia sinensis, or Salvia miltiorrhiza, or any combination thereof. For example, the combination composition can comprise a mixture of plant materials comprises Artemisia annua (sweet annie), Salvia miltiorrhiza (red sage), Houttuynia cordata (Houttuynia), Isatis indigotia (Isatis), Bupleurum chinense (Bupleurum), Cinnamomum cassia (Cinnamon), Citrus reticulata (tangerine peel), Glycyrrhiz uralensis (licorice), Coptis spp. (Coptis), Zingiber officinale (Ginger), or any combination thereof. In embodiments, Houttuynia cordata is replaced with Scutellaria baicalensis. Such combination compositions, for example, can have increased activity, such as increased synergistic anti-viral activity. See, for example, FIG. 5. In embodiments, the combination composition can comprise Arum palaestinum, Houttuynia cordata, Scutellaria baicalensis, Chrysanthemum indicum. In embodiments, the combination composition can comprise Arum palaestinum, Houttuynia cordata, Scutellaria baicalensis, Camellis Sinensis. In embodiments, the combination composition can comprise Arum palaestinum, Houttuynia cordata, Scutellaria baicalensis, Salvia Miltiorrhiza. In embodiments, the combination composition can comprise Artemisia annua, Salvia miltiorrhiza, Houttuynia cordata, Isatis indigotia, Bupleurum chinense, Cinnamomum cassia, Citrus reticulata, Glycyrrhiz uralensis, Coptis spp., and Zingiber officinale. In embodiments, Houttuynia cordata is replaced with Scutellaria baicalensis.

The term “botanical extract” or “plant extract” can refer to a product prepared by separating, by chemical or physical process, one portion of a plant from another. For example, a product prepared by separating, by chemical or physical process, any medicinally active portions of a plant from the inactive or inert components. The botanical extracts prepared according to the invention can be obtained by means of a solvent (i.e., a polar solvent or a non-polar solvent), or under pressure and/or heat.

The term “solvent” can refer to a substance that can be dissolved or dispersed in one or more substances.

In embodiments, the plant extracts can be provided by the use of a polar solvent. The term “polar solvent” can refer to a solvent that comprises dipole moments. For example, a polar solvent can be miscible with water and polar solvents. For example, a polar solvent can comprise chemical species in which the distribution of electrons between covalently bonded atoms is not even. For example, the polarity of solvents can be assessed by measuring any parameter known to those of skill in the art, including dielectric constant, polarity index, and dipole moment (see, e.g., Przybytec (1980) “High Purity Solvent Guide,” Burdick and Jackson Laboratories, Inc.). The polar extracts of the invention can comprise any percentage of polar solvent including, but not limited to, for example 1-10% polar solvent, 10-20% polar solvent, 20-30% polar solvent, 30-40% polar solvent, 40-50% polar solvent, 50-60% polar solvent, 70-80% polar solvent, 80-90% polar solvent and 90-100% polar solvent. Examples of polar solvents include but are not limited to ethyl alcohol (ethanol), butyl alcohol (butanol), methanol, water, acetic acid, tetrahydrofuran, N,N-dimethylformamide, dichloromethane, ethyl acetate, acetonitrile, dimethylformamide, dimethyl sulfoxide, acetone, or n-propanol.

In other embodiments, the plant extracts can be provided by the use of a non-polar solvent (i.e. non-polar extract). As used herein, “nonpolar” and “non-polar” can be used interchangeably. As used herein, the term “nonpolar solvent” can refer to a solvent comprising molecules that do not have an overall dipole. For example, the solvent comprises molecules comprising bonds between atoms with similar electronegativities (e.g. a carbon-hydrogen bond). For example, the nonpolar molecule comprises equal sharing of electrons between atoms or the arrangement of polar bonds leads to overall no net molecular dipole moment. The non-polar extracts of the invention can comprise any percentage of non-polar solvent, including but not limited to, for example, 1-10% non-polar solvent, 10-20% non-polar solvent, 20-30% non-polar solvent, 30-40% non-polar solvent, 40-50% non-polar solvent, 50-60% non-polar solvent, 70-80% non-polar solvent, 80-90% non-polar solvent, and 90-100% non-polar solvent. Examples of non-polar solvents include but are not limited to isooctane, hexane, pentane, benzene, chloroform, diethyl ether, hydrocarbons, cyclohexane, toluene, or 1,4-dioxane.

Hydrophobic molecules can be non-polar and thus can interact with (e.g. associate, aggregate, etc.) other neutral molecules and non-polar solvents. For example, nonpolar or hydrophobic molecules can interact through non-covalent interactions. For example, the non-covalent interaction is a van der Waals interaction. For example, the van der Waals interaction are London forces. Hydrophilic molecules can be polar and dissolve by water and other polar substances.

Thus, the plant extracts can be produced by any method known in the art including a polar extract such as a water (aqueous) extract or an alcohol extract (e.g., butanol, ethanol, methanol, hydroalcoholic, see for example Swanson R L et al., 2004, Biol. Bull. 206: 161-72) or a non-polar extract (e.g., hexane or isooctane, see for example, Ng L K and Hupe M. 2003, J. Chromatogr A. 1011: 213-9; Diwanay S, et al., 2004, J. Ethnopharmacol. 90: 49-55).

Regardless of the exact solvent employed, plant extracts can be made by placing a plant sample (e.g., leaves, seeds, or other part of the plant) in a mortar along with a small quantity of liquid (e.g., 10 ml of water, alcohol or an organic solvent for every 2 grams of plant sample) and grinding the sample thoroughly using a pestle. When the plant sample is completely ground, the plant extract is separated from the ground plant material, such as by centrifugation, filtering, cation-exchange chromatography, and the like, and the collected liquid can be further processed if need be (such as by a concentrating column and the like), active ingredients can be separated from this extract via affinity chromatography, mass chromatography and the like.

The term “aqueous extract” can refer to a plant extract where the extraction has been performed using water as the only solvent. The term “organic extract” can refer to a plant extract where the extraction has been performed using an organic solvent that is not an alcohol. The term “alcoholic extract” can refer to a plant extract where the extraction has been performed using a mixture of water and an alcohol, such as methanol or ethanol. In another embodiment, the extract is a dry extract.

For example, the alcoholic extract can be about 10% alcohol extract, about 20% alcohol extract, about 30% alcohol extract, about 40% alcohol extract, about 50% alcohol extract, about 60% alcohol extract, about 70% alcohol extract, about 80% alcohol extract, about 90% alcohol extract, or about 99% alcohol extract.

For example, the alcoholic extract can be about 10%-30% alcohol extract, about 20%-40% alcohol extract, about 30%-50% alcohol extract, about 40%-60% alcohol extract, about 50%-70% alcohol extract, about 60%-80% alcohol extract, about 70%-90% alcohol extract, or about 80%-100% alcohol extract.

For example, the alcoholic extract can be an ethanolic extract, such as a 20% ethanol extract, or a 70% ethanol extract. For example, Arum, Salvia miltiorrhiza, Citrus reticulata, Glycyrrhiza uralensis, Coptis spp., and Salvia can be extracted in 10%-40% ethanol, such as 20% ethanol. Houttuynia, Scutellaria Chrysanthemum, Artemisia annua, Isatis indigotica, Bupleurum chinense, Cinnamomum cassia, Zingiber officinale, and Camellia can be extracted in 50%-100% ethanol, such as 70% ethanol.

In embodiments, the botanical composition can comprise a plant extract prepared from an entire plant, or a plant extract prepared from a part of a plant, such as flowers, flowering tops, aerial parts, leaves, stems, buds, roots, bulbs, rhizomes, bark, seeds, fruit or fruit peel, bark, kernel, stones, berries, sap, resin, latex and thallus of the plant.

The term “aerial part” can refer to those parts of the plant that exists above a solid surface, such as those parts that exists in the air or in the space above the solid surface. Non-limiting examples of aerial parts of a plant comprise stems, leaves, petioles, flowers, fruit or fruit peel, seeds, and bark.

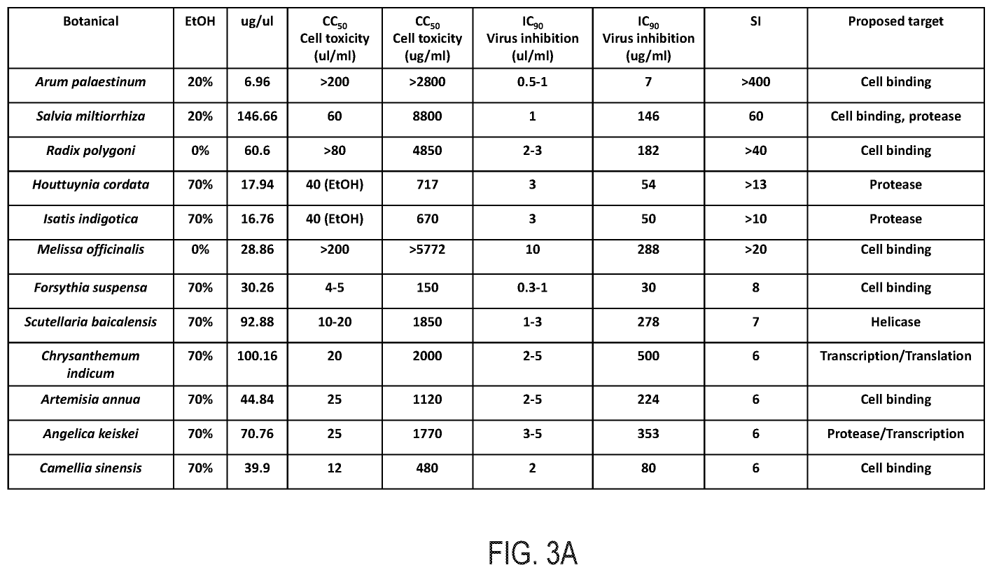

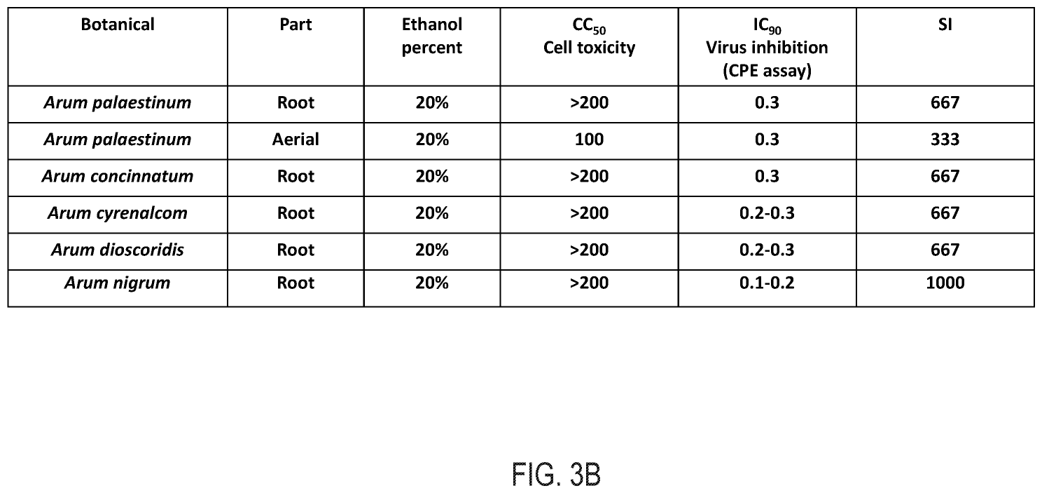

For example, the botanical composition can include a plant extract prepared from the root or aerial parts of the Arum genera plant. Referring to FIG. 3B, the species of the Arum genera have anti-viral activity. Arum is a genus of flowering plants in the family Arceae. In embodiments, the Arum genera plant comprises Arum palaestinum, Arum concinnatum, Arum cyrenalcom, Arum dioscoridis, Arum nigrum, or any combination thereof. For example, the plant extract can be prepared from the root or aerial parts of Arum palaestinum. For example, the plant extract can be prepared from the root parts of Arum concinnatum, Arum cyrenalcom, Arum dioscoridis, or Arum nigrum. For example, the plant extract can be prepared from the root and/or aerial parts of Arum palaestinum, Arum concinnatum, Arum cyrenalcom, Arum dioscoridis, or Arum nigrum. In embodiments, the plant extract prepared from the Arum genera plant can be present in the botanical composition in an amount ranging from about 1 to about 50 wt % of the total composition, for example about 1 to about 25 wt % of the total composition, such as about 1 to about 20 wt % of the total composition.

The botanical composition can also include a plant extract prepared from Houttuynia cordata. Houttuynia cordata is one of two species in the genus Houttuynia (the other being H. emeiensis). For example, the plant extract can be prepared from the aerial parts of Houttuynia cordata, such as a leaf. In embodiments, the plant extract prepared from Houttuynia cordata can be present in the botanical composition in an amount ranging from about 1 to about 50 wt % of the total composition, for example about 1 to about 25 wt % of the total composition, such as about 5 to about 20 wt % of the total composition.

The botanical composition can further include a plant extract prepared from Scutellaria baicalensis. Scutellaria baicalensis, also known as Huang Qin or Chinese skullcap, is species of flowering plant in the family Lamiaceae. For example, the plant extract can be prepared from the root of Scutellaria baicalensis. In embodiments, the plant extract prepared from Scutellaria baicalensis can be present in the botanical composition in an amount ranging from about 15 to about 80 wt % of the total composition, such as from about 30 to about 70 wt % of the total composition.

Botanical compositions as described herein can further comprise one or more additional plant extracts. For example, such one or more additional plant extracts can be prepared from Chrysanthemum indicum, Camellia sinensis, Salvia miltiorrhiza, or any combination thereof.

Chrysanthemum indicum is a flowering plant within the family Asteraceae. For example, the plant extract can be prepared from the flower parts of Chrysanthemum indicum. In embodiments, the plant extract prepared from Chrysanthemum indicum can be present in the botanical composition in an amount ranging from about 25 to about 75 wt % of the total composition, such as about 50 to about 60 wt % of the total composition

Camellia sinensis is a species of evergreen shrubs or small trees in the flowering plant family Theaceae. For example, the plant extract can be prepared from the leaf parts of Camellia sinensis. In embodiments, the plant extract prepared from Camellia sinensis can be present in the botanical composition in an amount ranging from about 5 to about 50% wt of the total composition, such as about 15 to about 25 wt % of the total composition.

Salvia miltiorrhiza is a perennial plant in the family Lamiaceae. For example, the plant extract can be prepared from the root parts of Salvia miltiorrhiza. In embodiments, the plant extract prepared from Salvia miltiorrhiza can be present in the botanical composition in an amount ranging from about 20 to about 60 wt % of the total composition, such as about 30 to about 40 wt % of the total composition.

Artemisia annua is a type of wormwood plant in the family Asteraceae, which is also known as sweet annie. In embodiments, the plant extract prepared from Artemisia annua can be present in the botanical composition in an amount ranging from about 5% to about 70% of the total composition, such as about 15% to about 35% of the total composition.

Isatis indigotica is a flowering plant in the family Brassicaceae. In embodiments, the plant extract prepared from Isatis indigotica can be present in the botanical composition in an amount ranging from about 1% to about 40% of the total composition, such as about 5% to about 30%.

Bupleurum chinense is a plant in the family Apiaceae. In embodiments, the plant extract from Buplerum chinense can be present in the botanical composition in an amount ranging from about 1% to about 30%, such as about 2.5% to about 25%.

Cinnamomum cassia is an evergreen plant in the Lauraceae family that can also be referred to as cinnamon. In embodiments, the plant extract of Cinnamomum cassia can be present in the botanical composition in an amount ranging from about 0.25% to about 10%, such as about 2.5% to about 7.5%.

Citrus reticulata is a citrus fruit tree in the Rutaceae family that can also be referred to as tangerine peel. In embodiments, the plant extract of Citrus reticulata can be present in the botanical composition in an amount ranging from about 1% to about 20%, such as about 2.5% to about 15%.

Glycyrrhiza uralensis is a flowering plant in the Fabaceae family, which can also be referred to as licorice. In embodiments, the plant extract of Glycyrrhiza uralensis can be present in the botanical composition in an amount ranging from about 1% to about 20%, such as about 2.5% to about 15%.

Coptis spp. are a group of species in the Coptis genus which belong to the Ranunculaceae family and the Coptidoieae subfamily. The Coptis genus comprises approximately 10-15 flowering plant species. In embodiments, the plant extract of Coptis spp. can be present in the botanical composition in an amount ranging from about 0.25% to about 20%, such as about 1% to about 15%.

Zingiber officinale is a flowering plant belonging to the Zingiberaceae family, which can also be referred to as ginger. In embodiments, the plant extract of Zingiber officinale can be present in the botanical composition in an amount ranging from about 0.25% to about 15%, such as about 1% to about 5%.

In embodiments, the botanical composition can comprise plant extracts from Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale. For example, the botanical composition can comprise Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale in a ratio of 12:8:6:4:4:4:4:1:1. For example, the botanical composition can comprise Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale in a ratio of 19:4:11:11:16:4:11:11:11:2. For example, the botanical composition can comprise Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale in a ratio of 31:6:19:19:5:5:5:5:2.5:2.5.

In embodiments, the botanical composition can comprise plant extracts from an Arum genera plant, Houttuynia cordata, and Scutellaria baicalensis. For example, the botanical composition can comprise plant extracts from the Arum genera plant, Houttuynia cordata, and Scutellaria baicalensis in a ratio of about 1:3:3.

Embodiments can also comprise botanical compositions comprising plant extracts from the Arum genera plant, Houttuynia cordata, Scutellaria baicalensis, and Chrysanthemum indicum. For example, the botanical composition can comprise plant extracts from the Arum genera plant, Houttuynia cordata, Scutellaria baicalensis, and Chrysanthemum indicum in a ratio of about 1:3:3:4.

Embodiments can further comprise botanical compositions comprising plant extracts from the Arum genera plant, Houttuynia cordata, Scutellaria baicalensis, and Camellia Sinensis. For example, the botanical compositions can comprise plant extracts from the Arum genera plant, Houttuynia cordata, Scutellaria baicalensis, and Camellia sinensis in a ratio of about 1:3:3:2.

Still further, embodiments can comprise botanical compositions comprising plant extract from the Arum genera plant, Houttuynia cordata, Scutellaria baicalensis, and Salvia miltiorrhiza. For example, the botanical compositions can comprise plant extracts from the Arum genera plant, Houttuynia cordata, Scutellaria baicalensis, and Salvia miltiorrhiza in a ratio of about 1:3:3:1.

The botanical compositions can be prepared simply by mixing any combination of the plant extracts (e.g., Arum genera plant, Houttuynia cordata, Scutellaria baicalensis) described herein together with one or more pharmaceutically acceptable excipients. Such combinations (i.e., one or more plant extracts and a pharmaceutically acceptable excipient) can be referred to as a “therapeutic botanical preparation”. For example, a therapeutic botanical preparation can be used for prevention, treatment, control or slow down a disease and/or its symptoms. In embodiments, a therapeutic botanical preparation can include plant extracts from an Arum genera plant, Houttuynia cordata, and Scutellaria baicalensis, and at least one pharmaceutically acceptable excipient. In embodiments, a therapeutic botanical preparation can include plant extracts from Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale, and at least one pharmaceutically acceptable excipient. In embodiments, Houttuynia cordata is replaced with Scutellaria baicalensis. Therapeutic botanical preparations can further comprise one or more additional agents, such as one or more additional plant extracts. In embodiments, the one or more additional plant extracts can comprise a plant extract from Chrysanthemum indicum, Camellia sinensis, Salvia miltiorrhiza, or a combination thereof.

In this way, the composition can be formulated for administration via several conventionally acceptable routes. For example, the botanical composition can be provided in a form selected from the group consisting of a liquid, a gummy, a tablet, a tincture a pellet, a lozenge, a granule, a suspension, and a capsule.

In embodiments, the botanical composition can be prepared by mixing the active plant extracts with one or more inert excipients (e.g., carriers, vehicles, binders, diluents etc.) suitable for the selected route of administration. The term “excipients” can refer to pharmaceutically acceptable organic or inorganic substances which do not deleteriously react with the active compounds. Examples of excipients include, but are not limited to, dietary suitable starch, vegetable oil, vegetable gums, gelatins, soy extracts, sugars, grains, natural and artificial flavorings, and the like. Examples of carriers include, but are not limited to, water, salt solutions, alcohol, plant seed and vegetable oils, glycols, gelatin, lactose, amylose, magnesium stearate, talc, silicic acid, viscous paraffin, perfume oil; fatty acid monoglycerides and diglycerides, fatty acid esters, hydroxymethylcellulose, and the like. The botanical compositions described herein can also be formulated as syrups and elixirs. Further, additional compositions can be readily prepared using technology which is known in the art such as described in detail in Remington's Pharmaceutical Sciences, Twentieth Edition.

The botanical compositions can be in a form suitable for oral use, for example, as tablets, troches, lozenges, pills, aqueous or oily suspensions, solutions, dispersible powders or granules, emulsions, hard or soft capsules, syrups or elixirs, pastes, gels or the like. Compositions intended for oral use can be prepared according to any known method, and such compositions can contain one or more agents selected from the group consisting of sweetening agents, flavoring agents, coloring agents, and preserving agents in order to provide commercially viable, pharmaceutically elegant and palatable compositions. Tablets can contain the active ingredient(s) in admixture with non-toxic pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They also can be coated for controlled delivery. For example, a “delayed release” dosage form releases a product or substance at a time other than promptly after administration. Examples of delayed-release systems include repeat-action tablets and capsules, and enteric-coated tablets where timed release is achieved by a barrier coating.

The terms “carrier oils” can refer to any lipid-based carrier materials, oil and/or aqueous solution, derived from any plant seed sources, suitable for administration of the active compounds in the botanical compositions. Carrier oils useful herein include any such materials known in the art that are nontoxic, have stand alone beneficial and therapeutic effects, and do not interact with other components. The term “a pharmaceutically acceptable carrier” can refer to any substantially non-toxic carrier conventionally useable for administration in which the active composition of the invention and its derivatives will remain stable and bioavailable.

Botanical compositions can be formulated as oily suspensions in a pharmaceutically acceptable carrier such as a soft gelatin capsule whereby the active ingredient(s) is (are) mixed with an aqueous solution or oil medium, including suspending the active ingredient in a plant seed derived or vegetable oil, for example hemp seed oil, evening primrose seed oil, borage seed oil, olive oil, sesame oil or coconut oil. The oily suspensions can contain a thickening agent or other agent necessary to produce a commercially viable product, such as, beeswax, paraffin, lecithin, or cetyl alcohol. Sweetening and flavoring agents can be added to provide a palatable oral composition. These compositions can be preserved by the addition of an antioxidant such as ascorbic acid.

Compositions of the invention can be sterilized and/or mixed with auxiliary agents and other excipients including but not limited to other carrier oils, preservatives, glycerins, stabilizers, waxes, wetting agents, emulsifiers, suspending agents, lecithin, esters or partial esters, buffers, coloring agents, flavorings and/or aromatic substances and the like which do not deleteriously react with the active compounds.

The combinations of plant extracts into a botanical composition can produce a synergistic effect/action. “Synergy” can refer to an effect produced by a combination (e.g., of two or more plant extracts) that is greater than the expected additive effectives of the combination components. In embodiments, “synergy” between two or more plant extracts can result in increased anti-viral activity. As used herein, “synergy” or “synergistic interactions” can refer to the interaction or cooperation of two or more plant extracts to produce a combined effect greater than the sum of their separate effects.

Referring to FIG. 5, for example, the synergistic combination can comprise a mixture of plant extracts comprising Arum genera (e.g., Arum palaestinum), Houttuynia cordata, Scutellaria baicalensis, and one or more of Chrysanthemum indicum, Camellia sinensis, or Salvia miltiorrhiza. The combination of the three botanicals Arum palaestinum, Houttuynia cordata, Scutellaria baicalensis, plus either Chrysanthemum indicum, Camellia sinensis, or Salvia miltiorrhiza, for example, resulted in increased synergistic anti-viral activity.

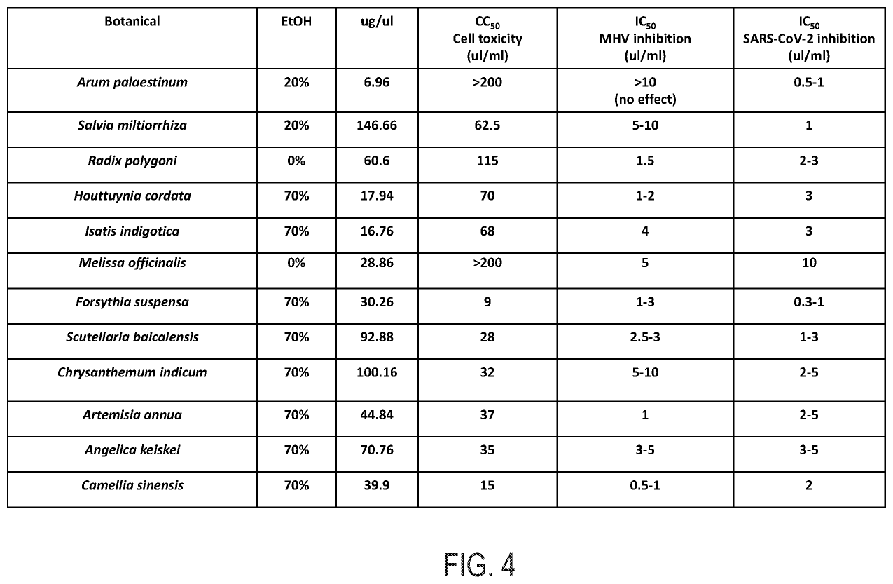

Referring to FIG. 3A, for example, the synergistic activity can result from combinations of botanical extracts that have different mechanisms of action. For example, the mechanism of action targeted by botanical extracts include, but are not limited to, cell binding, protease, helicase, and transcription/translation. In embodiments, many of these targets in the viral replication process are complex and can have multiple unique targets for each replication step. For example, Arum spp. and Camellia sinensis can both target viral cell binding. However, Arum spp. only inhibited SARS-CoV-2 (not MHV) while C. sinensis effectively inhibited both SARS-CoV-2 and MHV (FIG. 4). This indicates that each botanical extract can have constituents that target different aspects of viral attachment to the cell, and thus can provide positive synergistic activity related to this step in the viral replication process.

Referring to FIG. 6, for example, the synergistic combination can comprise a mixture of plant extracts comprising Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale. In embodiments, Houttuynia cordata is replaced with Scutellaria baicalensis.

Botanical compositions as described herein further comprise aqueous botanical extract compositions. For example, the aqueous botanical extract composition can comprise a combination of plant extracts, such as from Arum genera plant (i.e., Arum palaestinum, Arum concinnatum, Arum cyrenalcom, Arum dioscoridis, Arum nigrum, or a combination thereof), Houttuynia cordata, Scutellaria baicalensis. For example, the aqueous botanical extract composition can comprise a plant extract prepared from the Arum genera plant present in the aqueous botanical extract composition in an amount ranging from about 1 to about 50 wt % of the total composition, for example about 1 to about 25 wt % of the total composition, such as about 1 to about 20 wt % of the total composition. For example, the aqueous botanical extract composition can comprise a plant extract prepared from Houttuynia cordata present in the aqueous botanical extract composition in an amount ranging from about 1 to about 50 wt % of the total composition, for example about 1 to about 25 wt % of the total composition, such as about 5 to about 20 wt % of the total composition. For example, the aqueous botanical extract composition can comprise a plant extract prepared from Scutellaria baicalensis present in the botanical composition in an amount ranging from about 15 to about 80 wt % of the total composition, such as from about 30 to about 70 wt % of the total composition.

Botanical compositions as described herein further comprise an aqueous botanic extract composition comprising a combination of plant extracts, such as plant extracts from Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale. In embodiments, the Artemisia annua is present in an amount ranging from about 15 to about 40 wt % of the total composition. For example, the aqueous botanical extract composition can comprise a plant extract prepared from Salvia miltiorrhiza present in an amount ranging from about 1 to about 30 wt % of the total composition. For example, the aqueous botanical extract composition can comprise a plant extract prepared from Isatis indigotica present in an amount ranging from about 5 to about 30% wt of the total composition. For example, the aqueous botanical extract composition can comprise a plant extract prepared from Bupleurum chinense present in an amount ranging from about 1 to about 30 wt % of the total composition. For example, the aqueous botanical extract composition can comprise a plant extract prepared from Cinnamomum cassia present in an amount ranging from about 0.5 to about 10% wt of the total composition. For example, the aqueous botanical extract composition can comprise a plant extract prepared from Glycyrrhiza uralensis present in an amount ranging from about 1 to about 20% wt of the total composition. For example, the aqueous botanical extract composition can comprise a plant extract prepared from Coptis spp. present in an amount ranging from about 0.25 to about 20% wt of the total composition. For example, the aqueous botanical extract composition can comprise a plant extract prepared from Zingiber officinale present in an amount ranging from about 0.25 to about 10% wt of the total composition. In embodiments, Houttuynia cordata is replaced with Scutellaria baicalensis.

Aqueous botanical extract compositions can further comprise one or more additional plant extracts, such as from Chrysanthemum indicum, Camellia sinensis, and/or Salvia miltiorrhiza. For example, the aqueous botanical extract composition can comprise a plant extract prepared from Chrysanthemum indicum present in the aqueous botanical extract composition in an amount ranging from about 25 to about 75 wt % of the total composition, such as about 50 to about 60 wt % of the total composition. For example, the aqueous botanical extract composition can comprise a plant extract prepared from Camellia sinensis present in the aqueous botanical extract composition in an amount ranging from about 5 to about 50% wt of the total composition, such as about 15 to about 25 wt % of the total composition. For example, the aqueous botanical extract composition can comprise a plant extract prepared from Salvia miltiorrhiza present in the aqueous botanical extract composition in an amount ranging from about 20 to about 60 wt % of the total composition, such as about 30 to about 40 wt % of the total composition.

Methods of Treatment

Aspects of the invention are also drawn to methods for preparing a liquid extract (i.e., a plant extract) from a plant material. Such plant extracts can be used in the botanical compositions described herein. For example, the methods described herein can be used for preparing a liquid extract (i.e., a plant extract) from a plant material selected from an Arum genera plant, Houttuynia cordata, Scutellaria baicalensis, Chrysanthemum indicum, Camellia sinensis, and Salvia miltiorrhiza. For example, the methods described herein can be used for preparing a liquid extract (i.e., a plant extract) from a plant material selected from Artemisia annua, Salvia miltiorrhiza, Houttuynia cordata, Isatis indigotica, Bupleurum chinense, Cinnamomum cassia, Citrus reticulata, Clycyrrhiza uralensis, Coptis spp., and Zingiber officinale. In embodiments, Houttuynia cordata is replaced with Scutellaria baicalensis.

The methods for preparing a liquid extract (i.e., plant extract) from a plant material comprises obtaining plant material; grinding the plant material, such as to a powder; combining the powder with a liquid; and extracting the plant material to form a liquid extract.

In embodiments, the phrase “obtaining a plant material” can refer to any direct or indirect method of obtaining, securing, receiving, etc. a plant material. For example, the plant material can be fresh plant material, that has not been processed or has been minimally processed.

Optionally, the plant material can be washed and/or dried prior to grinding the plant material. For example, the plant material can be washed with a solution, such as water or water with a low percentage of bleach. For example, the method comprises drying the plant material, at least partially, or fully. According to an embodiment drying the plant material can be conducted at about ambient temperature in an environment of controlled humidity. According to an embodiment, said plant material is dried to moisture content of less than 30% wt, less than 20% wt, less than 15% wt or less than 10% wt.

Grinding the plant material can be accomplished by, for example, plant material grinding equipment or other similar equipment known in the art, such as a pestle, hammer, cylinder mill (such as a ball mill, a rod mill, etc.), a roller mill (such as a wheel mill, a ball roller mill, etc.), a disk mill (such as a rotary crusher, an attritor, a colloid mill, etc.), and an impact mill (such as a hammer mill, a micro mill, etcroller, ball, jaw, rotary, or other similar equipment). Grinding of the plant material can be accomplished utilizing equipment according to the particle size of the intended ground product.

The product of the grinding step, such as a powder, can be combined the solid product with a liquid (i.e., an extractant) to produce a liquid composition for extracting. For example, the powder can be combined with at least one of water and ethanol to concentrate the solution to obtain an alcohol extract of the plant materials.

For example, the alcoholic extract can be about 10% alcohol extract, about 20% alcohol extract, about 30% alcohol extract, about 40% alcohol extract, about 50% alcohol extract, about 60% alcohol extract, about 70% alcohol extract, about 80% alcohol extract, about 90% alcohol extract, or about 99% alcohol extract.

For example, the alcoholic extract can be about 10%-30% alcohol extract, about 20%-40% alcohol extract, about 30%-50% alcohol extract, about 40%-60% alcohol extract, about 50%-70% alcohol extract, about 60%-80% alcohol extract, about 70%-90% alcohol extract, or about 80%-100% alcohol extract.

The term “extracting” can refer to separating into another phase. For example, extracting can refer to separating components from a solid material, e.g. provided solid plant material, into a liquid phase. Alternatively, extracting can refer to separating components from a solid material into a vapor phase, and optionally condensing said vapors.

As used herein the term “contacting a solid material with and extractant” can refer to any form of contacting, e.g. washing the solid with the extractant, mixing the two or driving the extractant through a column containing the solid to produce a liquid extractant.

In embodiments, the extracting step can be performed at about 20° C. to about 37° C. to form a liquid extract. For example, the mixture can be agitated for a period of time (such as 24 hours at room temperature (˜25-30° C.), followed by centrifugation at 4000×g for 10 min to remove solid plant material. The supernatant (extract) was then filtered through a 0.2 uM filter to sterilize. The skilled artisan will recognize that any sterilization technique can be used to sterilize the extract, such as filtration, high heat (e.g., autoclave), or UV irradiation.

Once the solid plant material has been contacted with (i.e., combined with) the extractant, the liquid phase (i.e., liquid extract) can be separated from the remaining solid phase (i.e., plant material), thereby producing a plant extract.

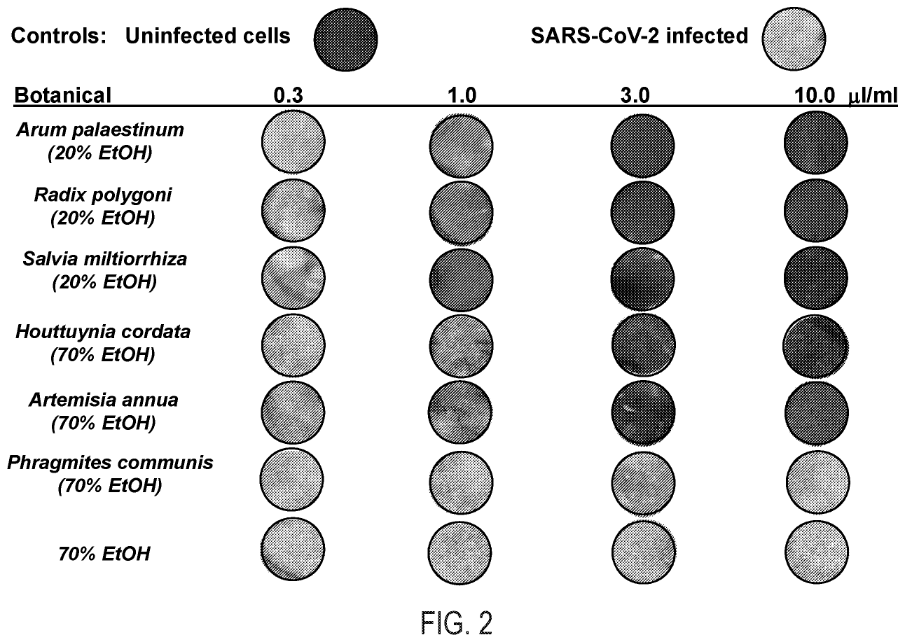

Aspects of the invention are further directed to methods for preventing, treating, or alleviating a symptom or complication of a disease or disorder caused by coronavirus, for example, a Severe Acute Respiratory Syndrome coronavirus (SARS-CoV2). Referring to FIG. 2, for example, plant extracts such as those from Arum palaestinum, Radix polygoni, Salvia miltiorrhiza, Houttuynia cordata, and Artemisia annua have anti-viral activity against SARS-CoV-2. Referring to FIG. 4, for example, plant extracts from these and other plants have anti-viral activity against MHV, a murine coronavirus.

The term “virus” can refer to a submicroscopic infectious agent that replicates inside of living cells of an organism. For example, the virus can refer to a coronavirus. The term “coronavirus” can refer to a virus whose genome is plus-stranded RNA of about 27 kb to about 33 kb in length depending on the virus. The virion RNA has a cap at the 5′ end and a poly A tail at the 3′ end. The length of the RNA makes coronaviruses the largest of the RNA virus genomes. In one embodiment, coronavirus RNAs encode: (1) an RNA-dependent RNA polymerase; (2) N-protein; (3) three envelope glycoproteins; plus (4) three non-structural proteins. These coronaviruses infect a variety of mammals & birds. They cause respiratory infections (common), enteric infections (mostly in infants >12 mo.), and neurological syndromes. Coronaviruses can be transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), SARS-coronavirus (Center for Disease Control), human coronavirus NL63 (HCoV-NL63), severe acute respiratory syndrome coronavirus (SARS-CoV), Middle East respiratory syndrome coronavirus (MERS-CoV), or severe acute respiratory syndrome coronavirus-2 (SARS-CoV-2).

In embodiments, the corona virus can be a severe acute respiratory syndrome coronavirus (SARS-coronavirus) The terms “SARS-CoV”, “SARS” or “SARS-associated Coronavirus” can refer to any strain of coronavirus associated with severe acute respiratory syndrome.

SARS-CoV-2 is a recently discovered human pathogen that is a member of the betacoronavirus genus. Infection with SARS-CoV-2 can result in disease and has led to a global pandemic. For example, the viral infection or viral disease can refer to coronavirus disease 19 (COVID-19). COVID-19 and SARS-CoV-2 can be used interchangeably. Infection with SARS-CoV-2 can lead to symptoms such as fever, severe respiratory illness, and pneumonia, some symptoms are so severe as to result in death (Wrapp et al. Science 13 Mar. 2020, 367(6483) 1260-1263). As some of the most severe SARS-CoV-2 symptoms affect the respiratory system, as well as several other organs, and with no currently known cure, a treatment for respiratory symptoms, and those resulting from other affected organs, induced by a virus or prevention of viral infection is a need.

The phrase “viral infection” or “viral disease” can be used interchangeably, and can refer to an infection, disease, or disorder caused by both RNA and DNA viruses and can refer to any stage of viral infection, including incubation phase, latent or dormant phase, acute phase, and development and maintenance of immunity to a virus. A “viral infection” or “viral disease” can be characterized by a strong correlation between exposure to a virus and the development of pathological changes, and that the pathological changes have an immune mechanism (i.e., a viral inflammatory response). For example, the immune mechanism can refer to leukocytes exhibit an immune response to viral stimulation. For example, the immune response can refer to increased production of pro-inflammatory cytokines and chemokines.

Aspects of the invention are drawn towards methods of decreasing the viral activity of a coronavirus. As used herein, “viral activity” can refer to the ability of a virus to replicate, multiply, reproduce or infect a cell or a subject. Referring to FIG. 3A, embodiments of the invention can target viral activities such as cell binding, protease, helicase, and/or transcription/translation.

For example, embodiments comprise methods of decreasing replication of a virus. As used herein, virus “replication” can refer to the process that results in the production of a copy of the genetic material of a virus. Virus replication, and changes in virus replication, can be determined by measuring standard signs related to virus replication in a cell. Examples include, but are not limited to, cytopathic effects, virus titers, amount of viral polynucleotide, hemagglutination, nucleoprotein, viral load in an animal's sinuses, lungs, trachea, and/or pathology associated with infection by a virus. Assays for measuring viral replication are known in the art and include, but are not limited to, plaque assays, TCID50, hemagglutination inhibition assays, immunofluorescence, quantitative real time polymerase chain reaction (RT-qPCR), and release of the virus from a cell.

In embodiments, the methods herein comprise administering to a subject a therapeutic botanical composition as described herein. The term “subject” or “patient” can refer to any organism to which aspects of the invention can be administered, e.g., for experimental, diagnostic, prophylactic, and/or therapeutic purposes. For example, subjects to which botanical compositions can be administered include animals, such as mammals. Non-limiting examples of mammals include primates, such as humans. A wide variety of subjects are suitable for veterinary applications, such as livestock such as cattle, sheep, goats, cows, swine, and the like; poultry such as chickens, ducks, geese, turkeys, and the like; and domesticated animals for example pets such as dogs and cats. A wide variety of mammals are suitable subjects for diagnostic or research applications, such as rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

As used herein, “treatment” and “treating” can refer to the management and care of a subject for the purpose of combating a condition, disease or disorder, such as a viral infection, viral disease, or viral induced inflammatory response, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

The phrase “alleviating a symptom of” can refer to ameliorating, reducing, or eliminating any condition or symptom associated with a viral infection, viral disease, or viral induced inflammatory response. Non-limiting examples of symptoms of viral infection, viral disease or viral induced inflammatory response comprise high viral loads, respiratory distress, and pulmonary damage correlated with high cytokine abundance. Cytokines coordinate the body's response to infection, trigger inflammation, and in COVID-19 (SARS-CoV-2) they can be generated in uncontrolled amounts. Generation of uncontrolled amounts of cytokines can be referred to as a “cytokine storm”. The term “cytokine storm” can refer to a series of events that result in a devastating and sometime fatal immune reaction that comprises a positive feedback loop between cytokines and immune cells that in turn leads to highly elevated levels of various cytokines. Cytokines that are induced during cytokine storm include, e.g., one or more of the following: IL4, IL2, IL1β, IL12, TNF, IFNγ, IL6, IL8, and IL10. Cytokine storm can lead to multi-organ failure (heart, lung, kidneys) and lead to death. Non-limiting examples of symptoms of viral infections, for example SARS-CoV-2, include cough, shortness of breath, difficulty breathing, fever, chills, muscle pain, headache, exhaustion, sore throat, loss of taste or small, nausea, vomiting and/or diarrhea. Symptoms can appear 2, 5, 14, 28, or greater than 28 days after exposure to the virus.

Aspects of the invention are further drawn to compositions and methods of treating, ameliorating, or preventing disease associated complications. For example, the disease associated complication is a viral disease associated complication. For example, the disease associated complication is a coronavirus disease associated complication. In embodiments, disease associated complications can refer to an unfavorable result of a disease, health condition, or treatment. For example, a disease associated complication can adversely affect the prognosis, or outcome, of a disease.

In embodiments, the method comprises treating or alleviating a viral infection related complication. In embodiments, the viral infection is SARS-CoV2. In embodiments, the viral infection related complication and/or symptom is hypoxemia, hypoxia, hypercoagulation, respiratory distress, respiratory difficulty, or end organ damage. For example, hypoxemia can refer to an insufficiency of oxygen in the blood or a tissue. For example, hypercoagulation can refer to an increase in coagulation tendency. For example, an increase in coagulation tendency can refer to an increased risk of excessive thrombus formation. Non-limiting examples of conditions that can result from hypercoagulation include pulmonary embolism, heart attack, and stroke.

An aspect of the invention is also directed to methods of preventing or delaying the onset of one or more symptoms of a coronavirus associated disease or condition. For example, a coronavirus associated disease or condition comprises lung pathology, acute respiratory distress syndrome, pneumonia, organ failure, heart failure, blood clots, acute kidney injury, and/or additional viral or bacterial infections.

As an example of a coronavirus associated disease or condition, acute respiratory distress syndrome can refer to when fluid collects in alveoli, depriving organs of oxygen. Non-limiting examples of symptoms of acute respiratory distress syndrome and respiratory distress comprise shortness of breath, increased breathing rate, skin color changes, nostril flaring, and wheezing, labored and rapid breathing, low blood pressure, confusion and tiredness.

Aspects of the invention are also drawn to methods of ameliorating a symptom of a disease or disorder caused by a coronavirus. “Ameliorating a symptom” can refer to an improvement of at least one discernible symptom or at least one measurable physical parameter of a disease or disorder. For example, symptoms of viral infection include stuffy or runny nose, sore throat, cough, congestion, body aches, headaches, sneezing, fever, and general malaise. Aspects of the invention are further drawn towards preventing or delaying the onset of symptoms.

Embodiments described herein comprise administering to a subject a therapeutically effective amount of a botanical composition described herein. The term “therapeutically effective amount” as used herein can refer to that amount of an embodiment of the botanical composition or plant extract therein administered that will relieve to some extent one or more of the symptoms of the viral disease, infection, or condition being treated, and/or that amount that will prevent, to some extent, one or more of the symptoms of the viral disease, infection, or condition that the subject being treated has or is at risk of developing. In one embodiment, the “effective amount” can refer to the amount of an agent that [ ].

The term “administering” or “administration” can refer to introducing a botanical composition described herein into a subject. Any route of administration can be utilized in embodiments herein, including but not limited to intranasal, topical, oral, intranasal, parenteral, intravitreal, intraocular, ocular, subretinal, intrathecal, intravenous, subcutaneous, transcutaneous, intracutaneous, intracranial, intravenous, peritoneal, intra-arterial, inhalation, vaginal, rectal, introduction into the cerebrospinal fluid, intravascular (such as in veins or arteries), or instillation into body compartments. In embodiments, “administering” can also refer to providing a therapeutically effective amount of a botanical composition to a subject.

A therapeutic botanical composition can be formulated to be compatible with its intended route of administration, such as those described herein. In any case, the therapeutic botanical composition to be administered can contain a quantity (e.g., therapeutically effective amount) of the botanical composition necessary to treat the subject.

The therapeutic botanical composition can be administered alone, but can also be administered with other compounds, such as excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; tinctures, lozenges, capsules; tablets; granules; pellets; powders; suspensions, emulsions, or microemulsions; patches; micelles; liposomes; vesicles; implants, including microimplants; eye drops; other proteins and peptides; synthetic polymers; microspheres; or nanoparticles.

Oral compositions can include an inert diluent or an edible carrier. They can be enclosed in gelatin capsules or compressed into tablets. For the purpose of oral therapeutic administration, the active compound can be incorporated with excipients and used in the form of tablets, lozenges, troches, or capsules. Oral compositions can also be prepared using a fluid carrier, such as for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In embodiments, the therapeutic botanical composition can be administered to a subject prior to exposure to the coronavirus, or after exposure to the coronavirus. For example, administration of the therapeutic botanical composition to a subject prior to exposure to the coronavirus is meant to prevent infection with the coronavirus, or delay the onset of symptoms of the coronavirus infection. In other embodiments, the therapeutic botanical composition can be administered to the subject with or shortly after the initial diagnosis of a viral disease or infection, such as of COVID-19, so as to prevent the further development of complications, worsening of symptoms, and/or enhancing the efficacy of anti-viral or immunotherapeutic treatments. Embodiments described herein can also be used in patients hospitalized with active viral disease or infection, patients in intensive care units.

Multiple doses of the pharmaceutical composition can be administered to the subject. The frequency of administration of the pharmaceutical composition can vary depending on any of a variety of factors, e.g., severity of the symptoms, the viral disease or infection being treated, and/or characteristics of the subject being treated. For example, in an embodiment, the pharmaceutical composition can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), four times a day, weekly, monthly, quarterly, semi-annually, annually, or by another regulator interval.

The method can further comprise administering to the subject one or more additional active agents, such as an anti-inflammatory agent, a pain reliever, an anti-viral drug, a viral entry inhibitor, a viral attachment inhibitor, or a combination thereof. Anti-viral agents can include lopinavir, ritonavir, or remdesivir, for example. In embodiments, a “pain reliever” can refer to an agent that can provide relief from pain. In embodiments, an anti-inflammatory agent can refer to an agent, compound, composition, or drug that can help reduce inflammation. Anti-inflammatory agents can include but are not limited to nonsteroidal anti-inflammatory drugs (NSAIDs), apremilast, icatibant, tocilizumab, sarilumab, or cenicriviroc. Second active agents can also comprise medications such as systemic corticosteroids, antibiotics, anticoagulants, convalescent sera or monoclonal antibodies. In embodiments, the composition is administered prior to or after exposure to a virus, such as SARS-CoV2.

Therapeutic Kits

A “kit” or “medical kit” can comprise a dosage form of a botanical composition described herein. A kit can include the botanical composition along with other active ingredients, such as in combination (e.g., in a single tablet), or provided separately, such as in two tablets.

Kits can further comprise one or more additional active agents. For example, the additional active agent can comprise a plant extract, an anti-viral agent, an anti-inflammatory agent, a pain reliever, a steroid, an inhibitor of fatty acid uptake, an inhibitor of fatty acid oxidation, or a combination thereof. In embodiments, the additional active agent comprises medications such as remdesivir, systemic corticosteroids, antibiotics and anticoagulants, convalescent sera or monoclonal antibodies. Kits can further comprise devices that are used to administer the active ingredients. Kits can also comprise printed instructions for administering the compound to a subject.

Kits can further comprise pharmaceutically acceptable excipients that can be used to administer one or more active ingredients. For example, if an active ingredient is provided in a solid form that must be reconstituted prior to administration, the kit can comprise a sealed container of a suitable vehicle in which the active ingredient can be dissolved to form a particulate-free sterile solution that is suitable for administration..

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1—Development of a Synergistic Botanical Extract Formulation for the Inhibition of SARS-CoV-2 and Other Coronavirus Replication

This technology is directed towards botanical therapeutics against coronavirus family members, for example SARS-CoV-2 and Mouse Hepatitis Virus (MHV). From 32 different botanicals tested, at least 12 botanical extracts were found to have potent anti-SARS-CoV2 activity. Without wishing to be bound by theory, this technology can fill a need for therapeutics to treat viral infections from the coronavirus family which remain limited in availability.

As described herein, botanical extracts were evaluated based on their mechanism of action. In an embodiment, a combination of four botanical extracts were found to have an increased synergistic anti-viral activity when compared to each botanical extract alone. Exemplary synergistic anti-coronavirus formulations include:
1) Arum spp., Houttuynia cordata, Scutellaria baicalensis, Chrysanthemum indicum (relative extract ratio of 1:3:3:4)
2) Arum spp., Houttuynia cordata, Scutellaria baicalensis, Camellia sinensis (relative extract ratio of 1:3:3:2)
3) Arum spp., Houttuynia cordata, Scutellaria baicalensis, Salvia miltiorrhiza (relative extract ratio of 1:3:3:1)

Botanical extracts were tested against another member of the coronavirus family, MHV, indicating extracts can have broad activity against different coronaviruses

### Example 2

### Example 2—Development of a Synergistic Botanical Extract Formulation for the Inhibition of SARS-CoV-2 and Other Coronavirus Replication

Botanicals have a rich tradition related to their therapeutic value. A multitude of pharmaceutical drugs have their origins from botanical sources. As described herein, 32 different botanicals were extracted under varying conditions and tested for their anti-viral activity against the coronavirus family members, SARS-CoV-2 and Mouse Hepatitis Virus (MHV). The most effective of these botanical extracts were evaluated based on their mechanism of action. Combinations of these botanicals in groups of four revealed the combination of the three botanicals Arum palaestinum, Houttuynia cordata, Scutellaria baicalensis, plus Chrysanthemum indicum, Camellia sinensis, or Salvia miltiorrhiza to have increased synergistic anti-viral activity attributable to different anti-viral mechanisms of action.

Coronavirus outbreaks represent a significant threat to the human population. Previous outbreaks with SARS-CoV-1 and MERS and more recently, SARS-CoV-2 illustrate the concerns over this virus family. Therapeutics to these viral infections remain limited. Described herein is the anti-coronavirus activity of multiple botanical extracts, including four that when combined together have increased anti-viral activity when compared to each botanical extract alone. This combination of botanical extracts represents of new therapeutic for the treatment of coronavirus infections.

Methods:

Thirty-two botanicals were selected to be tested for activity against the coronaviruses, SARS-CoV-2 and MHV. The botanicals tested and the part of the plant used are listed in FIG. 1. The botanicals were dried and then ground to a fine powder using a Vitamix blender. Each botanical was extracted in 70% ethanol/30% water or 20% ethanol/80% water at a ratio of 1:10 (weight:volume). The extraction mixture was mixed by constant rotation (30 rpm) for 24 hours. The solid plant material was then separated by centrifugation at 4,000×g for 5 minutes. The supernatant was filtered through a 0.2 μm filter. The filtrate was stored at room temperature and represents the final botanical extract to be tested.

The SARS-CoV-2 virus was obtained from BEI Resources and the strain used was USA-WA1/2020. The anti-SARS-CoV-2 activity was measured by a standard inhibition of viral cytopathic effect (CPE) assay. Confluent monolayers of Vero cells (maintained in DMEM, 10% FBS) were prepared in 12-well dishes. The virus was diluted to 100 plaque forming units (pfu) in 200 μl media. The diluted virus samples were treated with increasing doses of each botanical extract. This combined solution was then used to infect the Vero cell monolayer for 1 hour. Following the 1 hour infection, 1 ml of media was added to each well containing the same concentration of the botanical extract. The dishes were then incubated at 37° C. for 3 days. After three days, the media was removed and the cell monolayer stained with crystal violet. The level of viral CPE was estimated to determine the IC90 value (dose of the extract that inhibited viral CPE by 90%). An example of the CPE assay results are shown in FIG. 2.

In addition to determining the anti-viral activity for each botanical extract, the dose of each extract to induce cellular toxicity was determined. This assay was performed using a commercially available MTS Cell Proliferation Assay Kit (Abcam). This assay is a colorimetric sensitive quantification of viable cells based on the reduction of MTS tetrazolium compound by viable cells to generate a colored formazan product that is soluble in cell culture media. Vero cells were seeded into 96-well microtiter plates in a final volume of 200 μL media/well. The cells were treated with increasing doses of the botanical extracts and incubated for 24 hours. 20 μL of the MTS reagent was added to each well MTS Reagent into each well and incubated for an additional 2-4 hours at 37° C. The OD=490 nm of each well was measured using a plate reader and the CC50 (dose required to induce 50% cell toxicity) for each botanical calculated.

Based on the IC90 and CC50 values for each botanical extract, the Selectivity Index (SI) of each extract can be demonstrated by dividing the CC50 value by the IC90 value. Without wishing to be bound by theory, this value is important since the higher the SI ratio, the more effective and safer a drug is during in vivo treatment for a given viral infection. The IC90, CC50 and SI for the most effective anti-SARS-CoV-2 botanical extracts is shown in FIG. 3. This figure also shows the optimal extraction solution (20% or 70% ethanol) for each botanical. The concentration of the non-volatile constituents present in each botanical extract is listed for standardization purposes.

To demonstrate the most effective SARS-CoV-2 botanical extracts to inhibit other members of the coronavirus family, a plaque reduction assay was performed using the coronavirus MHV-A59. L2 cells were seeded in 6-well dishes. The confluent cell monolayers were infected with 100 pfu of MHV-A59 in the presence of varying concentrations of the botanical extracts. After the 1 hour infection, the monolayers were overlayed with low melt agarose/media containing the same dose of the botanical extract. The infected cell monolayers were incubated at 37° C. for 2 days after which the monolayers were stained with crystal violet and the number of viral plaques counted.

Results:

Thirty-two botanicals were tested for potential anti-viral activity against the coronavirus SARS-CoV-2 (FIG. 1). Since the active constituents of botanicals vary based on their solubility in different solvents, the dried botanical material was extracted in both 20% and 70% ethanol. The in vitro assay to demonstrate the anti-viral activity of these botanical extracts was an assay to measure the inhibition of CPE induced by the virus. An example of this assay is shown in FIG. 2. As shown, SARS-CoV-2 infection of the cell monolayer results in killing of the majority of cells (shown by a light grey appearance of the cell monolayer. If the cell monolayer remains intact, the cell monolayer appears as a solid black color). As shown in FIG. 2 for example, the 20% Salvia miltiorrhiza extract inhibited viral CPE around 1 μl extract/ml media and a 70% ethanol extract of Houttuynia cordata inhibited viral CPE around 3 μl extract/ml media. Other botanical extracts, like Phragmites communis, did not inhibit viral CPE at any dose tested. In addition, a control assay with only 70% ethanol did not inhibit viral CPE at any dose tested.

Following testing of the 32 botanical extracts prepared in both 20% and 70% ethanol (or just water for select botanicals), the top twelve most effective botanical extracts are shown in FIG. 3A. These botanical extracts were selected as the most effective extracts based on a low dose required to inhibit SARS-CoV-2 replication (CPE) and a high dose required (or lack of) for cell toxicity. Without wishing to be bound by theory, the botanical extracts represent those that have therapeutic efficacy against SARS-CoV-2 infections. Since the Arum palaestinum root extract was one of the most effective anti-SARS-CoV-2 botanical extracts (as indicated by the highest SI value), additional botanically-related extracts were prepared to include A. palaestinum aerial/leaf extract and extracts from other Arum species including A. concinnatum, A. cyrenalcom, A. dioscoridis and A. nigrum. Based on results from A. palaestinum, these additional extracts were prepared in 20% ethanol. As shown in FIG. 3B, the A. palaestinum aerial/leaf extract had similar anti-SARS-CoV-2 activity as the A. palaestinum root, although there was an increase in cell toxicity. The other Arum species root extracts from A. concinnatum, A. cyrenalcom, A. dioscoridis and A. nigrum had similar, if not better, anti-SARS-CoV-2 activity then that of the A. palaestinum root extract (FIG. 3B).

The family of coronaviruses represent a continued threat against human health. The 2019 pandemic with SARS-CoV-2, as well as previous outbreaks with SARS-CoV-1 in 2002 and MERS in 2012, signify the importance of developing therapeutics with broad anti-coronavirus activity. The top botanical extracts which were effective against SARS-CoV-2 were also tested for anti-viral activity against mouse hepatitis virus (MHV), a member of the coronavirus family. As shown in FIG. 4, 83% of the botanical extracts tested had similar anti-viral activity (within a 2-fold difference) when comparing the dose required to inhibit MHV and SARS-CoV-2. This indicates that these botanical extracts can have broad activity against multiple members of the coronavirus family.

Without wishing to be bound by theory, mechanisms of action of each of the top botanical extracts is listed in FIG. 3A. These mechanisms of action target multiple steps in the replication cycle of the virus ranging from cell surface receptor binding to viral enzymes including viral proteolytic or helicase activity. Without wishing to be bound by theory, as a therapeutic, a combination of botanical extracts targeting different steps in the viral replication cycle will be more effective. Such a combination of botanical extracts can provide positive synergistic activity by inhibiting replication of the virus at sequential steps in the viral replication process. In addition, such synergistic botanical extract combinations will reduce the developed resistance to the combined therapeutic formula by the virus. Multiple combinations of the top SARS-CoV-2 botanical extracts were tested for positive synergistic activity. The botanical extracts were prepared according to FIG. 3A and combined at ratios relative to their IC90 values. As shown in FIG. 5, the combination of the three botanicals Arum palaestinum, Houttuynia cordata, Scutellaria baicalensis, plus Chrysanthemum indicum, Camellia sinensis, or Salvia miltiorrhiza resulted in increased synergistic anti-viral activity. In this figure, the expected IC90 dose was the summation of the IC90 of each individual botanical extract in the formulation. For example, the expected IC90 dose for the combination of Arum palaestinum, Houttuynia cordata, Scutellaria baicalensis, and Chrysanthemum indicum was 11 μl extract/ml media based on the IC90 of each individual botanical extract. When tested, the combined IC90 was 2 μl extract/ml media which represented 0.18 μl Arum palaestinum, 0.27 μl Houttuynia cordata, 0.27 μl Scutellaria baicalensis, and 0.36 μl Chrysanthemum indicum and a 5.5-fold increase in activity. Without wishing to be bound by theory, this observed synergistic increase in anti-viral activity is attributable to different anti-viral mechanisms of action.

Embodiments herein comprise synergistic anti-coronavirus formulations including, but not limited to:
1) Arum spp., Houttuynia cordata, Scutellaria baicalensis, Chrysanthemum indicum (relative extract ratio of 1:3:3:4)
2) Arum spp., Houttuynia cordata, Scutellaria baicalensis, Camellia Sinensis (relative extract ratio of 1:3:3:2)
3) Arum spp., Houttuynia cordata, Scutellaria baicalensis, Salvia miltiorrhiza (relative extract ratio of 1:3:3:1)

Based on FIG. 3A, the majority of these botanical extracts have different mechanisms of action. Although some of the botanicals in these synergistic formulations have similar mechanisms of action, many of these targets in the viral replication process are complex and can have multiple unique targets for each replication step. For example, Arum spp. and Camellia sinensis can both target viral cell binding (FIG. 3A). However, Arum spp. only inhibited SARS-CoV-2 (not MHV) while C. sinensis effectively inhibited both SARS-CoV-2 and MHV (FIG. 4). This indicates that each botanical extract can have constituents that target different aspects of viral attachment to the cell, and thus can provide positive synergistic activity related to this step in the viral replication process.

The family of coronaviruses represent a continued threat against human health. The 2019 pandemic with SARS-CoV-2, as well as previous outbreaks with SARS-CoV-1 in 2002 and MERS in 2012, signify the importance of developing therapeutics with broad anti-coronavirus activity. Botanicals represent a traditional source of therapeutics for most of the past century. Eleven percent of the 252 drugs considered essential by the World Health Organization are exclusively derived from flowering plants. For example, artemisinin, the active anti-malarial constituent from Artemisia annua, was awarded the 2015 Nobel Prize in Physiology or Medicine. Botanical medicine has been used by the human population for thousands of years. Even today, 75 to 85 percent of the world's population continues to rely on botanical medicines dispensed by traditional healers for primary healthcare. In the European Union alone, 100 million people routinely rely on the use traditional, complementary or botanical medicine.

With our research, we have screened several botanical extracts for anti-viral activity against SARS-CoV-2. From these tests, at least 12 botanical extracts were found to have potent anti-SARS-CoV2 activity. Additional testing of these botanical extracts against another member of the coronavirus family indicates that these botanical extracts can have broad activity against multiple members of the coronavirus family. Without wishing to be bound by theory, many of these botanical extracts target and inhibit viral replication at different points in the viral replication process. By combining these botanical extracts in different combinations, synergistic anti-viral activity was observed. As a therapeutic, these combinations of botanical extracts which target different steps in the viral replication cycle are more effective (than, for example, any extract alone). Without wishing to be bound by theory, combinations of botanical extracts can provide positive synergistic activity by inhibiting replication of the virus at sequential steps in the viral replication process. In addition, such synergistic botanical extract combinations will reduce the develop resistance to the combined therapeutic formula by the virus.

Therapeutics for the treatment of coronavirus infections are currently limited and often are directed toward supportive care for immune support or other physiological responses. Even fewer drugs are available which directly target the virus replication process. Our work has identified several botanical extracts with anti-viral activity against both SARS-CoV-2 and MHV coronaviruses. Specific combinations of these some of these botanical extracts demonstrated positive synergistic activity related to inhibition of the virus. Without wishing to be bound by theory, these synergistic botanical extract formulations have therapeutic value in preventing and/or treating SARS-CoV-2 and other coronavirus infections. In addition, these extracts can offer a resource for the isolation and identification of new anti-coronavirus compounds.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An oral botanical composition comprising plant extracts from an Arum genera plant, Houttuynia cordata, and Scutellaria baicalensis.

2. The botanical composition of claim 1, wherein the Arum genera plant comprises Arum palaestinum, Arum concinnatum, Arum cyrenalcom, Arum dioscoridis, Arum nigrum, or a combination thereof.

3. The botanical composition of claim 1, wherein the Arum genera plant selected from the group consisting of Arum concinnatum, Arum cyrenalcom, Arum dioscoridis, and Arum nigrum.

4. The botanical composition of claim 1 further comprising plant extract from Chrysanthemum indicum, Camellia sinensis, Salvia miltiorrhiza, or a combination thereof.

5. The botanical composition of claim 3 further comprising plant extract from Chrysanthemum indicum, Camellia sinensis, Salvia miltiorrhiza, or a combination thereof.

6. The botanical composition of claim 1, wherein the plant extract is an alcoholic extract.

7. The botanical composition of claim 6, wherein the alcoholic extract is a 20% ethanol extract.

8. The botanical composition of claim 6, wherein the alcoholic extract is a 70% ethanol extract.

9. The botanical composition of claim 1, wherein the plant extract from the Arum genera plant, Houttuynia cordata, and Scutellaria baicalensis are in a ratio of about 1:3:3.

10. An aqueous botanical extract composition comprising plant extracts wherein the composition comprises about 1-20% extract of Arum genera plant, about 5-20% Houttuynia cordata, and/or about 30-70% Scutellaria baicalensis.

11. The composition of claim 10 further comprising about 50-60% extract of Chrysanthemum indicum, about 15-25% extract of Camellia sinensis, and/or about 30-40% Salvia miltiorrhiza.

12. A therapeutic botanical preparation comprising plant extracts from an Arum genera plant, Houttuynia cordata, and Scutellaria baicalensis, and at least one pharmaceutically acceptable excipient.

13. The therapeutic preparation of claim 12, wherein the Arum genera plant comprises Arum palaestinum, Arum concinnatum, Arum cyrenalcom, Arum dioscoridis, Arum nigrum, or a combination thereof.

14. The therapeutic preparation of claim 12, wherein the Arum genera plant is selected from the group consisting of Arum concinnatum, Arum cyrenalcom, Arum dioscoridis, and Arum nigrum.

15. A method for preparing a liquid extract from a plant material selected from an Arum genera plant, Houttuynia cordata, Scutellaria baicalensis, Chrysanthemum indicum, Camellia sinensis, and Salvia miltiorrhiza, the method comprising:
obtaining fresh plant material;
washing and air drying the plant material;
grinding the plant material to a powder;
combining the powder with a liquid comprising at least one of water and ethanol to concentrate the solution to obtain an alcohol extract of the plant materials;
extracting plant material by liquid at about 20° C. to about 37° C. to form a liquid extract; and
separating the liquid extract from the plant material.

16. A method of treating a coronavirus infection in a subject in need thereof, the method comprising administering to the subject a botanical composition of claim 1.

17. The method of claim 16, wherein the coronavirus comprises human coronavirus NL63 (HCoV-NL63), severe acute respiratory syndrome coronavirus (SARS-CoV), Middle East respiratory syndrome coronavirus (MERS-CoV), or severe acute respiratory syndrome coronavirus-2 (SARS-CoV-2).

18. A method for decreasing coronavirus infection or reducing a symptom thereof in a subject in need thereof, the method comprising administering to a subject in need thereof an effective amount of the botanical composition of claim 1.

19. A method for preventing, treating or improving a coronavirus associated disease or condition, wherein the method comprises administering to a subject in need thereof the botanical composition of claim 1.

20. The method of claim 19, wherein the coronavirus associate disease or condition comprises lung pathology, acute respiratory distress syndrome, pneumonia, organ failure, heart failure, blood clots, acute kidney injury, and/or additional viral or bacterial infections.
